(19) United States
(12) Patent Application Publication
(10) Pub. No.: US 2018/0015300 A1
(43) Pub. Date: Jan. 18, 2018

(54) COACHING, DOCUMENTING, REPORTING, AND MANAGING SCALP HAIR RECOVERY WITH LASER PHOTO-THERAPY DEVICES

(71) Applicant: Theradome, Inc., Pleasanton, CA (US)

(72) Inventor: Tamim Hamid, Pleasanton, CA (US)

(21) Appl. No.: 15/713,657

(22) Filed: Sep. 24, 2017

Related U.S. Application Data
(63) Continuation-in-part of application No. 14/753,000, filed on Jun. 28, 2015, now Pat. No. 9,833,633.
Application No. 14/753,000 is a continuation-in-part of application No. 15/669,928, filed on Aug. 5, 2017.
(60) Provisional application No. 62/474,074, filed on Mar. 21, 2017.

(51) Int. Cl. A61N 5/06; A61B 5/00; A61B 17/00; A61N 5/067

(52) U.S. Cl. CPC A61N 5/0617; A61B 5/7425; A61B 5/0022; A61B 5/0077; A61B 5/4848; A61B 5/486; A61B 5/7465; A61B 5/0013; A61B 5/7246; A61B 2017/00221; A61N 2005/0647; A61N 2005/0652; A61N 2005/0663; A61N 2005/067

(57) ABSTRACT

Users of laser photo-therapy helmets are brought online into a larger community of other users and hair-loss professionals. Together, they assist and contribute to coaching one another in the best automated treatment regimens and use of the devices. Statistical and analytical data is centrally collected by network servers and web portals from the laser photo-therapy helmets through mobile app's installed on users' mobile devices. Data mining then makes it possible for a central operator to isolate which users are struggling and that could benefit from professional assistance. A statistical analysis also allows a variety of treatment regimens to be tested, and for the best ones that produce optimal results, to be identified and then widely promoted to the whole community.

### FIELD OF INVENTION

The present invention relates to a method of coaching users of laser photo-therapy devices to success by documenting, reporting, and managing their scalp hair recovery. And more particularly, to bringing together laser photo-therapy device users at home with hair-loss professionals in a community to work together more efficiently to improve the long-term results and user satisfaction with the laser photo-therapy.

### BACKGROUND

Hair-loss happens to all of us as we age, some more than others. Products to prevent and restore hair-loss offer hope, but disappointment looms if users expect too much or are too impatient for the lotions, shampoos, ointments, creams, and devices to have effect. Some therapeutic courses require as much as one hundred weeks to play out. However, the slow but steady progress and results can be very difficult for users to discern or recognize.

So, what is needed is a tool for users to receive coaching on the proper and regular use of devices and medicines, and to be reassured by self-documenting their progress in a series of time-lapse videos. Reassurances are vital in keeping users satisfied throughout their hair recovery journeys. And reassurances based on hard evidence help win the hearts and minds of professionals and therapists to continue to recommend the devices, methods, and medicines.

Traditionally, a wide variety of medicines and devices that regenerate and promote hair growth for the user-clients are sold by selected supplier-professionals that then provide follow up consulting and care. The number of user-clients worldwide can run into the millions.

Theradome Inc. markets a FDA-cleared laser photo-therapy device to treat hair-loss, e.g., their helmet model LH80 PRO. The Theradome LH80 PRO is a helmet-styled photo-therapy device that includes a microcomputer and control program, a low-level, coherent laser light diode array, and wireless transceiver access to connect with the Internet. Each device includes a unique serial number used to register the device through a mobile app with a central server. Hair-loss professionals that sell these devices also register with a web portal central server and are assigned oversight responsibilities organized by the device serial numbers they provided to retail users.

Users at home place the helmet on their heads periodically in scheduled sittings to receive a series of controlled scheduled doses of low-level coherent laser light at therapeutic wavelengths delivered to their scalps. Such helmet styled photo-therapy devices are fully described by me in U.S. patent application Ser. No. 14/753,000, filed Jun. 28, 2015, and my continuation-in-part (CIP) U.S. patent application Ser. No. 15/669,928, filed Aug. 5, 2017.

### SUMMARY

Briefly, embodiments of the present invention tie users of laser photo-therapy helmets into a larger community of other users and hair-loss professionals. Together, they assist and contribute to coaching one another in the best treatment regimens and use of the devices. Statistical and analytical data is centrally collected by network servers and web portals from the laser photo-therapy helmets through mobile app 106 installed on users' mobile devices. Data mining then makes it possible to isolate which users are struggling and that could benefit from professional assistance. A statistical analysis also allows a variety of treatment regimens to be tested, and for the best ones that produce optimal results, to be identified and then widely promoted to the whole community.

### SUMMARY OF THE DRAWINGS

FIG. 1 is a functional block diagram of a single user-client's part of a wider community organized online to coach, measurement, log, report, and manage hair loss recovery with low-dosage laser-light helmet devices like the Theradome LH80 PRO shown. The Diagram further shows therapeutic doses of 678-nm monochromatic laser light being delivered to the scalp of a user, a BLE wireless communication with a mobile device, and an installed ScalpCoach mobile app 106;

FIG. 2 is a diagram of the online network topology of a SCALPCOACH community 200 which includes many thousands of the single user-clients like that of FIG. 1. The web portals shown are accessible by conventional browsers on the Internet as webpages, or by the ScalpCoach mobile app 106 of FIG. 1;

FIG. 3 is a diagram of a table showing how several sub-functions in the programming and firmware must be distributed as front ends and back ends amongst the ScalpCoach mobile app 106, server, and web portal. The Diagram further represents the direction of dataflow between parts of each sub-function. The sub-functions listed here are not exhaustive, nor will all always be required;

FIGS. 4A-4E represent the several screens that can be displayed by the mobile device of FIG. 1 when a user is preparing to take a photo selfie of their scalp. FIGS. 4B-4E in particular represent the four different templates that can be superimposed over an otherwise conventional live viewfinder display of a smartphone in camera mode;

FIG. 5 is a diagram representing the choices a user is presented when navigating to the home page on the web portals;

FIG. 6 is a diagram representing the choices a user is presented when navigating to the web portals.

### DETAILED DESCRIPTION OF THE EMBODIMENTS

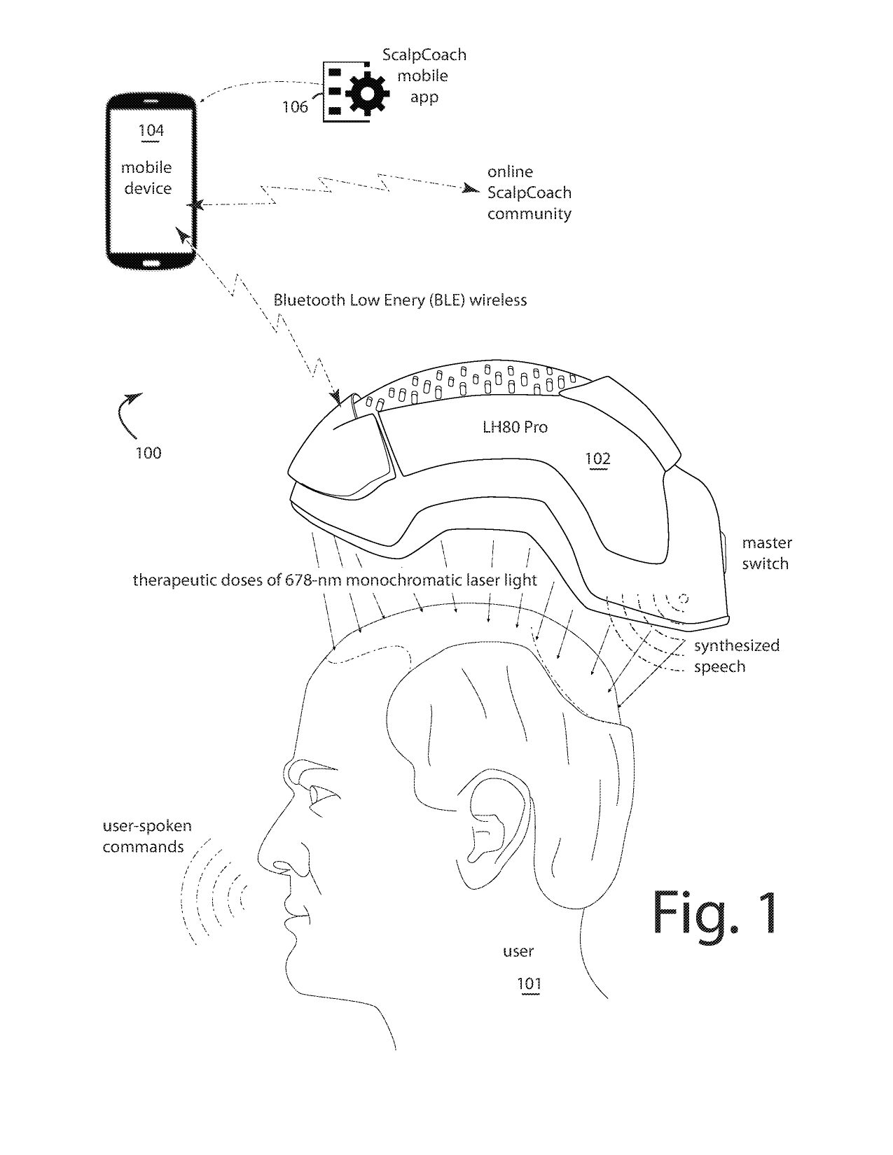

FIG. 1 represents a single-user site 100 in a much wider SCALPCOACH community 200 (FIG. 2) of thousands of users. The use of a laser light therapy hair restoration device begins with a treatment regimen. A balding single user 101 periodically wears a laser helmet (LH) 102 that emits therapeutic doses of 678-nm monochromatic laser light. Such doses are delivered in order to restore hairloss due to balding in prescribed amounts in up to one hundred weeks of daily-weekly treatment sessions. Voice commands can be spoken to LH 102 by user 101 with the assistance of a mobile device 104 and SCALPCOACH mobile app 106 and an online SCALPCOACH community 200 that provides artificial intelligence. Similarly, voice synthesized words and phrases can be spoken to user 101 by LH 102, also with the assistance of mobile device 104, SCALPCOACH mobile app 106, and the online SCALPCOACH community 200. Commands to LH 102 can be directly entered into mobile device 104 and SCALPCOACH mobile app 106. Status, logs, messages, and instructions from LH 102 can also be obtained directly from mobile device 104 with the coordination of functions provided by SCALPCOACH mobile app 106.

Each laser photo-therapy devices has an embedded microcomputer wirelessly connects to the Internet, such as through a Bluetooth Low Energy (BLE) transceiver. Each laser photo-therapy device is marked with a serial number the user-clients may use to register their particular laser photo-therapy device. Each laser photo-therapy device is thereafter able to report its usage statistics when it's online, and these statistics are collected and analyzed to provide and improve business management. For example, the collected usage statistics for user-client may indicate that their particular laser photo-therapy device isn't being used correctly, and so a push notification can be sent from the business manager to supplier-professional to follow up on that. Or, supplier-professional may discover the problem themselves on a routine survey of the several user-clients they're responsible for.

Should any registered user-client require customer support, they be directed to contact the customer help center and log in with their serial number. A complete record of the contact and the resolution are kept and used in statistical analyses to spot various operational difficulties the user-clients may be having with their individual laser photo-therapy devices. Such statistical analyses and the full context and content of the customer service contact are available to the administrator and selected bits to supplier-professionals who have a need-to-know.

A very similar, helmet-styled photo-therapy hairloss restoration device like LH 102 is more fully described by me in U.S. patent application Ser. No. 14/753,000, filed Jun. 28, 2015, and my continuation-in-part (CIP) U.S. patent application Ser. No. 15/669,928, filed Aug. 5, 2017. The online SCALPCOACH community 200 is more fully described herein, and also in my U.S. Provisional Patent Application 62/474,074, filed Mar. 21, 2017, and is one the present application claims benefit of. I am presently marketing my earlier versions of LH 102 commercially in the United States as the Theradome brand LH80 PRO, among other devices.

SCALPCOACH mobile app 106 is implemented for both iPhone and Android operating systems with CRM integration (account creation, helmet registration, chat function, store and customer login). Mobile app 106 includes functions for: Diagnosing hair-loss level; Tracking of progress through photos; Scheduling Theradome treatments and generating a schedule; Sharing of updates with hair-loss experts, family and friends through web portal, e-mail, social media and SCALPCOACH accounts; Getting feedback and guidance from hair-loss professionals; Inviting hair-loss professionals to join the SCALPCOACH community 200; Compiling reports and records for hair-loss professionals; Obtaining notifications and reminders for treatment sessions, updates on badges and rewards, blog updates, new products and other news; Earning gamification rewards; Entering monthly contests to win prizes; Accessing SCALPCOACH web portal 242 services through tight integration; Organizing photo galleries and time-lapse images.

Users are asked to input the start dates of their various hair and scalp regimens with frequency of use. A schedule is generated to reduce dosage for minoxidil and finasteride, and other medications likely to induce shedding. Theradome model LH80 PRO is set to ON as the default option. Users cannot proceed to a next section until a start date for treatments has been confirmed.

A “Photo Tutorial” is delivered to explain various camera template functions, set-ups and tips to for obtaining optimum photos results. Users are encouraged to enlist the help of another as photographer or to use the Theradome “ScalpSelfie”. The directions emphasize the importance of taking consistent, high-quality photos. Users take their first photo ScalpSelfies using one of four templates that each represent four head shots: 1) upper frontal, 2) upper rear, 3) right side, and 4) left side. Users can always return back to the Tutorial. Based on previous selections, a modal window introduces either a Savin or Norwood scale for female or male patients, respectively. Users are instructed to hold a “Compare” button on a subsequent screen to toggle between illustrations and their first photo. Closing the modal reveals the appropriate scale. Tapping a “Continue” button confirms a hair-loss scale selection.

Users can select 1:9 options for the Savin hair-loss scale. Users can hold the “Compare” button to toggle between the selected illustration and their first photo. Users then confirm their final selection by tapping a “Continue” button. Users can select 1:12 options for the Norwood hair-loss scale. Users can hold the “Compare” button to toggle between the selected illustration and their first photo. Users then confirm their final selection by tapping “Continue”. Users can select 1:6 options to determine their skin type. Skin type data does not affect a user's treatment and is only collected for internal reference. A treatment modal appears after all user data has been collected. Users are informed that a treatment schedule is about to be generated. Users proceed to set the first date and time of their treatment. Treatment schedule for a full year is generated based on a user's frequency settings. Treatment calendar has a “monthly” view as default, and a “weekly” view as an alternative. Users can thereafter share their treatment schedule, launch the camera for additional photos, and access all primary and secondary menu items. A first treatment can be scheduled by tapping dates on a treatment calendar that launches 1:4 modal screens. Users can either mark a treatment as completed or missed, or launch their calendar to modify or schedule other treatments.

Users can share dashboard reports, launch the camera to take additional photos, and access all primary and secondary menu items. Additional functions and choices include viewing data charts and infographics with widgets such as: “Optimal Energy Dosage Levels’: data chart that displays energy data for completed treatments; “Your Hair Loss Phase”: infographic to be updated based on number of completed treatments; “Discontinue Treatment”: counter that tabulates number of days to stop usage of other hair-loss treatments based on Proprietary usage; “Badge Counter Bar”: visual tracker for total number of badges earned; “Photo Counter Bar”: visual tracker for total number of photos taken; and “Next Scheduled Treatment”: digital timer to count down scheduled treatment. Users can share with a physician: SCALPCOACH account, Facebook, Twitter, e-mail or their own SCALPCOACH account by tapping the corresponding icons. One modal can be used for a treatment calendar, photo gallery, profile and rewards areas.

When taking photos after an initial photo, an updated photo template modal appears. Users are given five template choices instead of four, e.g., an additional overlay template option. The additional overlay template option allows users to superimpose live photos with previous photos to aid in consistent photo alignment and create comparison photos and time-lapse movies. Selecting a template launches the camera API. Users take their second photo using one of: upper frontal, upper rear, right side, left side and overlay templates. Users can opt at anytime to return to the dashboard. Users can share photo gallery reports, launch the camera to take additional photos, and access all primary and secondary menu items. Users can create comparison photos, time-lapse movies or access the photo tutorial. Photos are sorted by month with additional viewing options, e.g., initial and comparison photos or time-lapse movies.

Launching a “Compare Photo” widget asks the user to select two photos from a gallery. Photos are sorted by month, with additional options for viewing tagged photos, e.g., “upper frontal” and “upper rear”. Once confirmed, a composite comparison photo is added to the gallery area. Launching a “Time-lapse Movie” widget asks the user to select multiple photos from the gallery. Photos prior to display are sorted by month, with options for viewing tagged photos, e.g., “front top” and “back top”. Once confirmed, a time-lapse movie is then added to a main gallery area.

In summary, the SCALPCOACH mobile app 106: Allow users with mobile devices to communicate directly with their hair-loss experts; Provide extended functions to users; Provide private device level storage as well as access to SCALPCOACH web portal 242 250 and 260 (FIG. 2) for cloud storage; Allow users to access hair-loss treatment communities to confirm their results and progress; Facilitate the sharing of success results through the building of a dominant hair-loss community; Strengthen overall confidence in the technology; Become a dominant app for all patients involved in hair-loss treatment and/or experiencing hair-loss; Increase and maintain sales; Commercialize data collection; Commercialize a proprietary app; Further secure partners and customers; Improve product performance via usage models; Stay mainstream with the modern inclination to embrace technology.

FIG. 2 represents a SCALPCOACH community 200, and the communications, smartphone, computing, and networking equipment that interconnects them and that enables a coordinated and organized hair-loss treatment regimen for each individual helmet-styled photo-therapy hairloss restoration device like LH 102 (FIG. 1). Hair loss experts, salon owners, and other professionals 210, 220, and 230 sell or otherwise provide, manage, and coach users and their respective user clients 211-213, 221-223, and 231-233 in the proper use of devices like the LH 102.

The professionals 210, 220, and 230 receive assistance in their promotion of additional treatments and products to use in conjunction with the LH 102. This assistance helps with guarantees of future appointments, access to a client success dashboard, client retention after sale of the LH 102, sharing of practice success through data collection, and promotion of a local hair-loss practice through a clinic directory. Such assistance includes artificial intelligence automation and methods.

The user clients 211-213, 221-223, and 231-233 are individually assisted in their custom treatment regimens with: storing and tracking of progress photos they themselves take as selfies, connecting users with professionals for private consultations, accessing a help desk and support center, sharing treatments progress with physicians, friends and family, tracking past and future sessions, registering a LH 102 unit, scheduling of treatment reminders, chats with technical staff, purchases of complementary products, in earning points and rewards, and help in locating nearby hair-loss clinics.

Many of these client and professional functions are wholly provided or assisted by an Internet network server 240 with a SCALPCOACH Professional Web Portal 250, and a SCALPCOACH User Web Portal 260.

A systems administrator 270, such as a company business that has commercialized the LH 102, engages itself in data mining, business rules setting, and analytics for increasing revenue generation, retrieving marketing testimonials and progress photos, reading post-sales customer satisfaction and support statistics, opening user information dashboard, data mining usage models, customer behavior defined with Google analytics, gaining user insights to improve future products and product migration, and ways of increasing consumer confidence in hair-loss products.

Hair loss clients 211-213, 221-223, and 231-233 most likely wish to be discreet regarding their product use. They set up a treatment regimen schedule on SCALPCOACH web portal 242 260 to be reminded of individual treatment sessions. Clients generally use SCALPCOACH web portal 242 260 for: tracking overall LH 102 usage, taking photos and unlocking badges, creating a full hair-loss regimen profile, chatting with hair-loss experts if in need of troubleshooting or other type of help, ordering subscription-based complementary products, accessing treatment algorithms for the weening off of certain medications, such as minoxidil and finasteride, altering their treatment schedule, sharing photos with hair-loss experts, and through social media and hair-loss communities, managing the privacy of their SCALPCOACH web portal 242 posts, and participating in monthly giveaways with rewards based on usage criteria.

Clients usually buy the LH 102 independently and then can contact a hair-loss professional for support. Hair loss professionals 210, 220, and 230 can learn about LH 102 and incorporate the SCALPCOACH methods into their practice. Hair loss experts use the SCALPCOACH web portal 242 250 for: promoting their practice through a clinic directory and in generating new business from LH 102 users, viewing comprehensive patient histories, scalp photos and LH 102 schedules, viewing client experience postings, reviewing progress or lack thereof with the LH 102, viewing client progress photos and regimen of supplementary products with frequency of use, recommending additional products and treatments through the SCALPCOACH web portal 242, helping clients of other hair-loss medications withdraw which may no longer be necessary, adjusting the LH 102 treatment schedule of clients, increasing the visibility of client progress and the ability to schedule future appointments.

Clients connect with their respective hair-loss expert through the SCALPCOACH web portal 242 250 and 260 to keep them apprised of their progress through progress photos, treatment schedule, and their dashboard. Clients generally use SCALPCOACH for: Obtaining advice from hair-loss experts regarding progress, hair and scalp analysis, therapy level adjustment, addition (or weaning off) of medications; Sharing their progress through photos; Connecting with medical practitioners to schedule follow-up appointments; Contacting hair-loss experts with questions and concerns regarding their overall treatment; and Adjusting other medications with hair-loss experts taken for issues other than hair-loss.

Hair Restoration Clinic Clients planning on having a hair transplant and have usually established a relationship with a hair restoration clinic. The clinic may recommended using the LH 102 prior to and post-surgery to maximize success. Clients generally use SCALPCOACH for: Setting up their profile and registering their LH 102; Building their schedules based on the recommendations of their clinic; Taking and sharing progress photos with their clinic; Submitting progress photo updates and LH 102 usage; Sending weekly dashboards in adherence to the recommended regimen; Submitting progress dashboards in between appointments in order to set up future follow-ups and receive suggestions regarding their scalp regimen.

Salon Owner Clients are those that have purchased a LH 102 unit from their salon and are assisted by their stylist in their goals of hair restoration. These Clients generally use SCALPCOACH for: Obtaining guidance from their stylist and boosting their self-confidence; Taking frequent progress photos at home and in the salon; Sharing photos with their stylist; Obtaining tips from their stylist on how to maximize their success with Theradome; Getting new ideas for hair styling and suggestions for new products; and, Communicating directly with their stylist through the SCALPCOACH web portal 242.

Social Media Expert Clients that have suffered alopecia for a while and may have tried numerous products, such as topicals, shampoos, medications and vitamins, as well have adjusted their diet in an attempt to minimize hair-loss. These Clients generally use SCALPCOACH for: Linking up their profile and badge to all social media channels and setting up feeds to receive most up to date Theradome news; Alerting their followers of new progress photos and completed sessions; Following blog articles and reposting them through their own social media outlets; Sharing their earned badges and contest entries; and, Sending their dashboard and progress photos to their hair-loss experts to show their progress.

Technologically-Challenged Clients are overall very uncomfortable with technology. They have yet to register their LH 102 online, and are skeptical about using the SCALPCOACH regimen. These Clients generally use SCALPCOACH for: Ensuring they follow the default hair restoration regimen (FDA-cleared); Using calendar functions to remind them of scheduled treatment sessions; Reading blog articles and learning about updates; Staying informed regarding new products; and, Purchasing products.

System administrator 270 overseas a customer help center 271; all registrations 272 and system access control of all users, visitors, professionals, and clients; a statistical and analytics processor 273, and a business management automation 274.

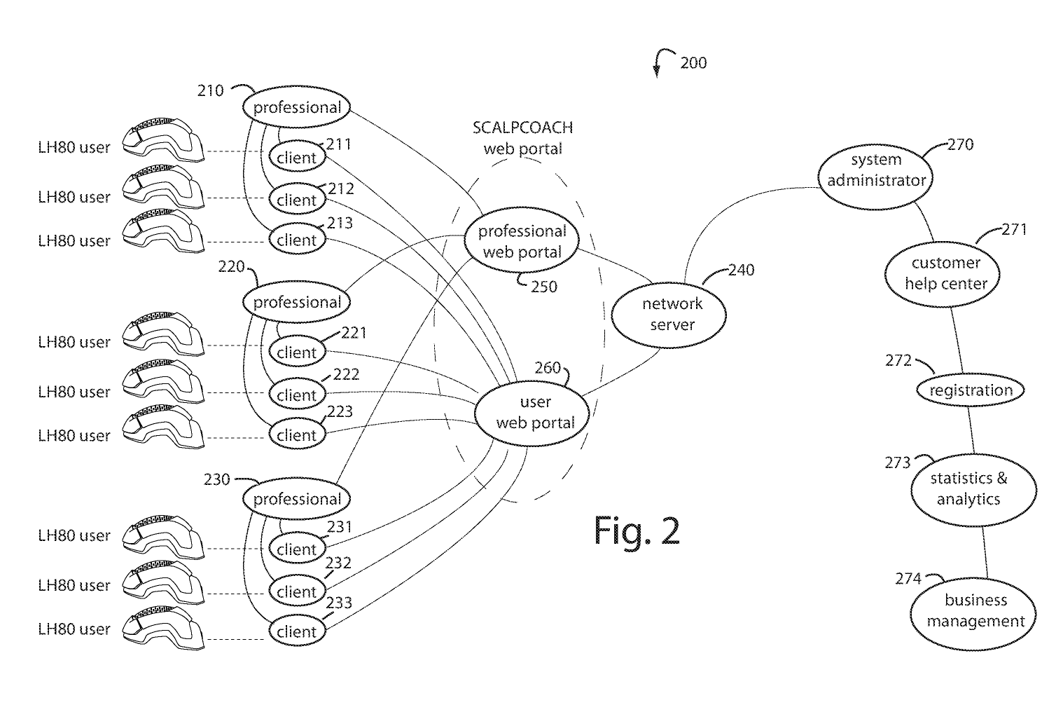

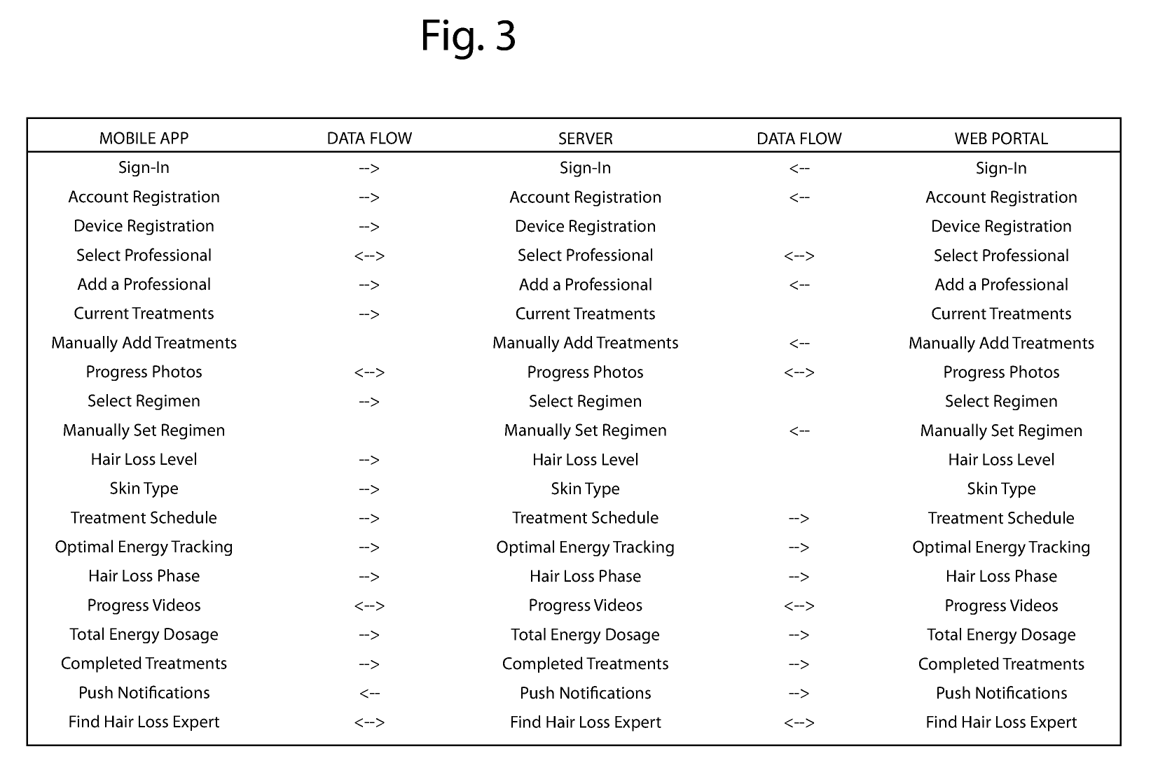

FIG. 3 represents the dataflow interplay between a mobile app 106 (in clients 211-213, 221-223, and 231-233), server 240, and SCALPCOACH web portal 242 250 and 260. Various left pointing, right pointing, and two-way arrows show which direction data flows between particular matched sub-functions.

FIG. 3 is actually a table that shows how several sub-functions in the programming and firmware must be distributed as front ends and back ends amongst the ScalpCoach mobile app 106, server, and web portal. The Diagram further represents the direction of dataflow between parts of each sub-function. The sub-functions listed here are not exhaustive, nor will all always be required. In software engineering, the terms front end and back end refer to the separation of concerns between the software's presentation layer (front end), and the data access layer (back end), or the equipment infrastructure and hardware. In a client-server model, each client is considered to be the front end, and the server is considered here to be the back end, even though some of the presentation work is done on the server.

For example, the following TABLE lists the basic sub-functions included in mobile app 106: Sign-In, Account Registration, Device Registration, Select Professional, Add A Professional, Current Treatments, Manually Add Treatments, Progress Photos, Select Regimen, Manually Set Regimen, Hair Loss Level, Skin Type, Treatment Schedule, Optimal Energy Tracking, Hair Loss Phase, Progress Videos, Total Energy Dosage, Completed Treatments, Push Notifications, and Find Hair Loss Expert.

TABLE

| Sub-Function | Description |
|---|---|
| Sign-In | User Validation with a user ID and password |
| Account Registration | First time registration of a new user |
| Device Registration | First time registration of a |
| | new laser therapy device |
| Select Professional | Select a professional to associate |
| | with from a local list |
| Add a Professional | Add a professional to associate with |
| Current Treatments | Treatments scheduled/completed |
| Manually Add | Treatments added to schedule |
| Treatments | |
| Progress Photos | Time-lapse series of photos of the users |
| | scalp documenting hair-loss recovery progress |
| Select Regimen | Select a standardized regimen from a list |
| Manually Set | Enter new regimen parameters |
| Regimen | |
| Hair Loss Level | Classify degree of hair loss |
| Skin Type | Classify user skin type |
| Treatment Schedule | Prescribed treatment schedule |
| Optimal Energy | Compare actual energy dose |
| Tracking | accumulations to a model |
| Hair Loss Phase | Classify which phase the hair loss is in |
| Progress Videos | Time-lapse movie built from photos |
| Total Energy Dosage | Compute total energy dose. |
| Completed | Log of completed treatments for this user/device |
| Treatments | |
| Push Notifications | News |
| Find Hair Loss Expert | Search for a Hair Loss Expert |

In general, embodiments of the present invention provide users an automated way to track their hair-loss treatment progress, and to connect hair-loss professionals with their clients and patients in a hair-loss community hosted online. The individual users are guided to collect progress photos with their mobile devices with voice synthesized instructions. These instructions help the users frame a series of consistent images into a smooth time-lapse series. Sharing these and other user-specific field data with hair-loss professionals enables optimized treatment plans to be dispensed and tracked. The laser photo-therapy helmets include a low-level coherent laser light diode array, a microcomputer and control program, and associated wireless access to connect with the Internet and the hair-loss community. Each laser photo-therapy helmet includes a unique serial number useful to register the device through a mobile app 106 with a SCALPCOACH web portal 242 central server. Hair-loss professionals that sell these devices also register with the SCALPCOACH web portal 242 central server. They are assigned oversight responsibilities according to the serial numbers of the laser photo-therapy helmets they sold to their retail users.

Method embodiments of the present invention collect and organize data from the devices, the mobile app 106, the central server, the users themselves, and the hair-loss professionals.

Mobile app 106 (FIG. 1) includes a sub-function that assists users in documenting their hair loss and their progress toward hair recovery with a series of “selfie” photos taken by mobile device 104. Each such selfie photo is confidentially retained, uploaded, and available to the professional community for analysis.

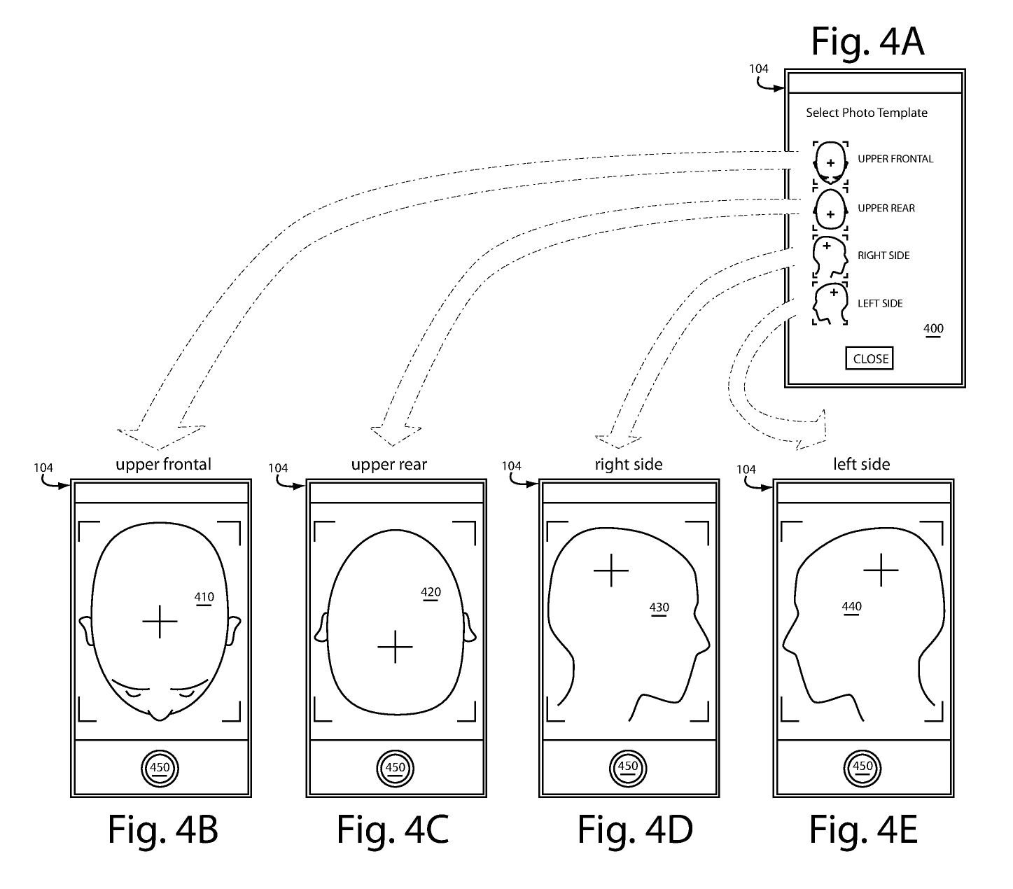

Referring now to FIGS. 4A-4E, a “Select Photo Template” screen 400 can be navigated to and displayed on mobile device 104 (FIG. 1). It allows the user to touch-select the camera for use with one of four templates, e.g., an upper frontal 410, an upper rear 420, a right side 430, and a left side 440. The digital images the built-in camera of mobile device 104 gets live are then automatically displayed behind the selected template. The purpose of the selected template is to help the user to properly frame the photo that will be “snapped” when a shutter-button 450 is touched.

This photo selfie sub-function advances the photo just taken most of the way to the state it ultimately needs to be in as a constituent frame in a time-lapse movie. A form of anti-jitter processing is employed to make small adjustments in each photo taken to remove any jump or jitter that would disrupt a smooth presentation. Anti-jitter camera photo frame processing for videos is conventional, and need not be described in further detail here.

It is obvious that a user would have some difficulty in taking such photos themselves. A selfie stick of my invention can help a user who has no one available to assist. I am commercializing such selfie stick under my brand name SCALPSHOT.

The SCALPSHOT selfie stick “pairs' with BLE wireless and the mobile app 106. It improves before and after photo taking which allows more comfort and consistent progress photos, and increases compliance and customer progress tracking for successful results. The SCALPSHOT selfie stick provides a telescoping 31” extension in a smooth and easy extendable handle. It is wireless; portable; mini-size, lightweight; and travel friendly. A silicone-coated universal-phone holder gripper holds mobile device 104 securely. The whole unit is one-piece with a built-in remote shutter that doesn't require a charger.

In summary, SCALPSHOT is a tool to simply using a mobile device with a camera to take a series of photographs of the users' scalps. It helps ensure each photograph is consistently framed and cropped using manual and electronic means before each is added to a time-lapse movie by a before-and-after auto moviemaker. Each time a user takes a photo of their scalp according to computer synthesized voice directions, the mobile app 106 automatically adds the latest photo. An objective observation of the success of the hair-loss therapy is therefore available during the course of treatments to the user and their responsible hair-loss professional.

SCALPCOACH portals 250 and 260 are directly accessible with a browser from the Internet. They provide Learning about the optimum treatments for hair-loss; Sharing and following the hair restoration journeys of other members; Uploading progress photos to public or private galleries for hair-loss expert analysis; Communicating as needed with hair-loss experts; Logging their hair-loss treatments and various scalp regimens; and Ordering and check-out for subscription-based complementary products.

A treatment progress photos repository includes: Comparison and time-lapse photo galleries; Communication Facility between clients and hair-loss professionals; Help desk and support; Progress sharing between physicians, friends, and family; Tracking past and future treatment sessions; Chat sessions with technical staff; Complementary product webstore; and, Hair-loss clinic directory.

PROPRIETARY USERS Creation of repository for treatment progress photos; Creation of comparison and time-lapse photo galleries; Facilitated process for ongoing communication between clients and hair-loss professionals; Access to help desk and support; Sharing of progress with physicians, friends and family; Ability to track past and future treatment sessions; Chat sessions with technical staff; Purchasing of complementary products; Access to hair-loss clinic directory.
HAIR-LOSS EXPERTS Improved client retention after sale; Implementation of a natural recruiting process from success cases; Promotion of additional treatments and products to use in conjunction; High potential of future appointments or call backs; Access to dashboard of client success; Validity of treatment regimen using the efficacy and convenience of the proprietary technology; Promotion of hair-loss practice with professional directory.
THERADOME Increased confidence level in the product brand for all individuals experiencing some hair-loss; Increased revenue generation through sales of laser helmets and complementary products; Establishment of a natural recruiting process by marketing customer cases of product success; Marketing of database of testimonials and progress photos; Availability of statistics regarding post-sales customer satisfaction and support; Enticement of hair-loss individuals to share data via a compelling dashboard; Data mining of usage models for hair-loss individuals; Establishment of dominant portal and marketplace for hair-loss communities; Access to user insights for future products and their improvement.

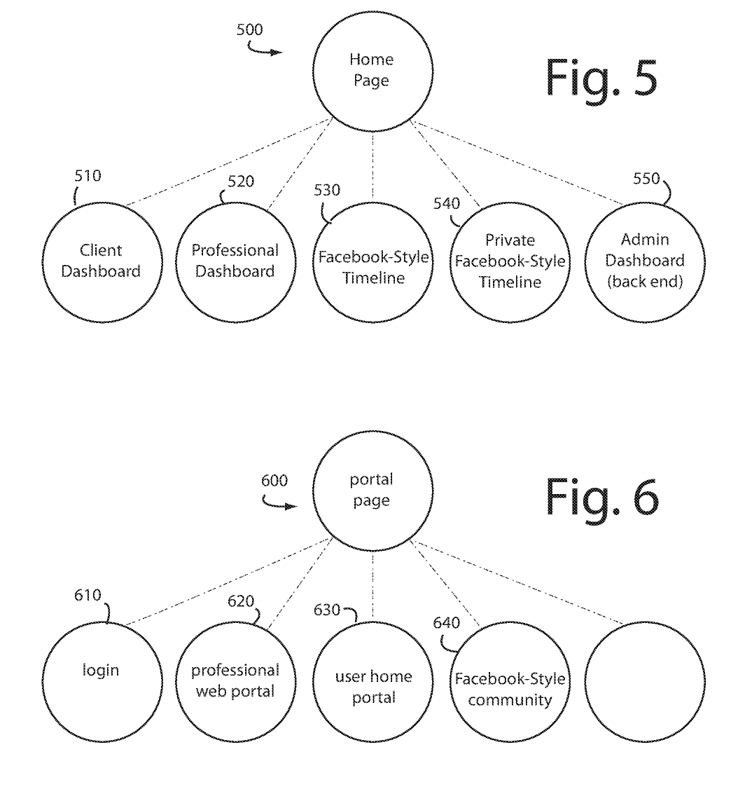

Referring now to FIG. 5, a SCALPCOACH web portal 242 comprises a home page 500 includes access to: an Overview of SCALPCOACH portal benefits and functions; Interface of Laser helmet use in conjunction with hair-loss medications; Sign-in and registration; Public galleries displaying hair restoration progress photos; “Total Clinical Photos Stored” counter; Current, updated user statistics infographics with totals for the number of Stored clinical photos, Clinical photos added daily, Registered SCALPCOACH portal users, and Number of countries with clients and hair-loss experts using SCALPCOACH portal; Link to terms and conditions; and Standard website footer and links.

A client dashboard 510 enables clients to: Tweak a user profile page (automatically created for SCALPCOACH registrants all others must register through portal); Set up account; Update user profile and report settings; Create optional public alias name; Create public or private timeline pages; Enable comments for public pages; Communicate with hair-loss professionals; View and print reports regarding visits to hair-loss experts; View, upload, download and share personal progress photos and videos on public or private pages; Share personal progress photos and videos with hair-loss experts; View publicly shared galleries created by other users; View current, updated progress data shared through SCALPCOACH; Create, view and edit personal profile, supplements, medications and topical treatments; and, Delete user profile.

A professional dashboard 520 allows hair-loss experts to: Set up an account with a verification of professional credentials using custom ID; Create user profiles and customize report settings; Browse through client gallery; View client data (dashboard, photos and treatment regimen); View and download client progress photos and videos; Share and print photos and videos; Access client accounts to directly post photos taken during their appointments; View client profile, supplements, medications and topical treatments; Keep private medical record of patient (not shared with client); View and print reports regarding their client visits; Delete user profile; Delete client accounts; Access patient demographic reports; Utilize a professional marketing module that allows the promotion of their own products and services; Utilize a marketing e-mail function to mass e-mail all of their clients or post on their individual account pages; and, Advertise their own practice.

A Facebook-style timeline 530 allows users to: Post timeline progress photos and videos; Comment on public postings; Share on social media; and, Advertise professional services or products.

A private Facebook-style timeline 540 allows users to: Post and share progress photos and videos with specific individuals or with closed communities; and, Enable private comments on these postings.

An admin dashboard 550 is used to: Set up user and hair-loss professional accounts; Access user profiles and reports; View profiles and galleries of all professionals and clients; View client progress data; View and download client progress photos and videos; Share and print photos and videos; View client profiles and their various scalp regimens; Delete hair-loss experts and clients user profiles; View and analyze demographic data, including age, gender, and hair-loss classification; View reports on screen; Export reports to different formats, e.g., PDF, Excel, CVS, etc.; Track rate of user abandonment and inactivity; Maintain, update and edit clinic directory; and, Verify credentials of the Community's hair-loss experts.

Referring now to FIG. 6, SCALPCOACH web portal 242 further comprise a portal page 600 that links to a login page 610, a professional web portal page 620, a user home portal page 630, and a Facebook-style community page 640.

Login page 610 page provides links for: Professional login option with verification of professional status using custom ID; User login; SCALPCOACH portal logo; Account set-up with Proprietary users carried over from SCALPCOACH, Public Users with basic info, e.g., contact name, e-mail, and scalp regimen. Hair-loss Professionals: login name, business/clinic address; Login and password; Description of SCALPCOACH portal; Login help if needed, such as the ability to recover password after three failed login attempts; and, Terms and conditions and privacy statement.

The professional web portal page 620 includes:
1) A client communication page with: most recent client uploaded progress photos; Client names with message titles and dates that progress photos were sent; Buttons to indicate whether a client needs a response or if an action is to be taken:
   Acknowledgment of client as patient
   Appointment requested for established patient
   Appointment requested for non-patient
2) A client page with: List view of all SCALPCOACH portal clients with the following:
   Client name
   Client e-mail
   Profile button to view client profiles in more detail
   Option to delete client from dashboard;
   Detailed dashboards with customizable client demographics and their individual scalp regimens.
3) Individual client pages have: Client contact information; Treatment types, scalp regimen and timeline; Information from SCALPCOACH community 200, e.g., absorbed energy dosage in Joules (J), treatment schedule, frequency of use; Galleries of progress photos and time-lapse videos with the ability to examine each in more detail; Tools to pair progress photos, add comments or markings for communication purposes; Reports of client demographics; History of reports generated for clients with the ability to print or view online; Tools to edit information; and Comprehensive selection of manageable infographics and progress charts.
4) A “creation of reports” function with: Generation of reports on practice/clinic letterhead; Selection of progress photos from gallery for comparison and analysis purposes with editing capabilities (labeling and photo mark-ups with graphic editor tools); Indication for notes below photos—can be typed or added by hand once printed; Description and information on client condition, along with supplemental treatment options, articles for benefit of client, and posttreatment care and notes; Ability to print reports and upload to client SCALPCOACH portal account; Comprehensive selection of manageable infographics and progress charts
5) A hall of success with: highlights of progress photos and success stories; Client testimonials; Positive reviews of professional practice; Sharing capabilities for promotion of practice; Accessibility from Professional Web Portal home page 500 (FIG. 5).
   A proprietary user home page 630 provides links to: All information from SCALPCOACH portal 242 (FIG. 2):
   Sortable progress photos and time-lapse videos
   Ability to compare photos side by side
   Total energy dosage absorbed in joules
   Treatment sessions completed/missed
   Future scheduled sessions
   Skin type
   Hair-loss level
   Hair-loss professional;
      Privacy settings; Facebook-style community timeline; Directory of hair-loss professionals; Reports from hair-loss experts; Ability to communicate with hair-loss professionals; Records of past communication with hair-loss professionals; Tools to edit personal information; Comprehensive selection of straightforward infographics and progress charts.

Public user home page with: Detailed scalp regimen with list of product types and individual frequency of use; Product descriptions with photos, e.g., bottle of finasteride; Sortable progress photos and time-lapse videos; Privacy settings; Facebook-style community timeline; Directory of hair-loss professionals; Reports from hair-loss experts; Ability to communicate with hair-loss professionals; Tools to edit personal information; Comprehensive selection of straightforward infographics and progress charts; Education module to learn more about Theradome.

Scrolling feed with: Progress updates from SCALPCOACH portal users; Advertisements for products; Advertisements for products sold by professionals; Articles from Proprietary blog and other reliable sources; Shared progress photos; Public dashboards.

In general, SCALPCOACH portal 242 provides laser helmet users a method to track their progress and to host an online hair-loss community that connect hair-loss professionals with clients/patients. Each Client/patient has the ability to connect with other SCALPCOACH clients/patients, track the other's progress photos, and hair-loss treatments. Such data sharing with hair-loss professionals enables their treatment plans to be optimized on the recommendation of professionals, as well as giving voice to effect hair-loss practices and product usage.

Hair growth is very difficult to track without before/after photos, people can be blind to the gradual changes to their hair recovery progress. Therefore, the SCALPCOACH tool is vital in keeping customers satisfied throughout their journeys, and encourages physicians to recommend the device. There is large community of hair-loss sufferers who frequent the SCALPCOACH portals.

Odoo can be used for an ecommerce and back end, and can fully integrate SCALPCOACH solutions within the Odoo framework.

One commercial objective is to sell as many laser helmets as possible with the greatest user and professional satisfaction levels. In support of this goal, the Primary advantages of embodiments of the present invention are:
Allow a Theradome customer/client to track and monitor results of their treatment;
Allow professionals and hair-loss experts to track a patient or client's treatment regimen, product use, and hair restoration progress;
Engage users with Theradome and build a strong customer service brand;
Ability to connect directly with hair-loss experts/customer service and sales teams;
Grow awareness and consult more readily between users/patients/clients and hair-loss professionals;
Notify customers of blog updates and new products;
Allow users to track their Theradome treatments;
Track customer patterns such as frequency of treatments, online purchases and use of supplementary medications;
Bring users together during their hair restoration process/journey;
Provide a mobile and cloud-based solution to taking and storing progress photos; and
Multiple variables such as medication/supplements, treatment regimen, medical procedures should all be included in the database for powerful research queries and reports for each and all [anonymous] users.

From an application perspective, the SCALPCOACH community 200 is implemented with two high level functional components, the SCALPCOACH web portal 242 and the mobile app 106. The SCALPCOACH web portal 242 is used by professionals and clients, and comprises Professional Web Portal 250 and User Web Portal 260.

User Web Portal 260 serves hair-loss product users, patients or clients of existing professionals, with an objective of educating public users about the benefits of our treatment regimen. The User Web Portal 260 presents several screens, the Client dashboard 510 being the primary screen.

User Web Portal 260 is configured for: 1) users who can alternatively access SCALPCOACH through the mobile app 106, 2) users who do not have smartphones, and 3) users who have not downloaded the mobile app 106 and can only create and update their account via SCALPCOACH community 200.

So, User Web Portal 260 provides users with practically the same tools as mobile app 106, e.g., in order to manage and share their treatment, including:
Modifying their profile page (which are automatically created for app registrants, all others must register through the portal);
Creating and setting up an account;
Updating their profile and report settings;
Creating an optional public username;
Creating public or private timeline groups;
Joining and commenting on public groups and timeline.

The SCALPCOACH community 200 helps with:
Communicating amongst hair-loss professionals;
Viewing and printing reports regarding visits to hair-loss experts;
Viewing, uploading, downloading and sharing personal progress photos and videos on public or private pages;
Sharing personal progress photos and videos with hair-loss experts;
Viewing publicly-shared galleries created by other users;
Viewing current, updated progress treatment data shared through SCALPCOACH community 200;
Creating, viewing and editing their personal profile and hair growth regimen, including supplements, medications and topical applications;
Tracking all hair-loss treatments;
laser helmet (LH) data uploaded from the helmet to SCALPCOACH;
Viewing, contributing to, creating, and joining groups on the SCALPCOACH community 200;
Connecting and chatting with a particular professional;
Rating their hair-loss professional; and
Deleting their user profile.

The Professional Web Portal 250 is provided for professionals and offers multiple screens, the Professional dashboard 520 is its primary screen. Professional Web Portal 250 provides tools to benefit their practice and manage clients, and includes:
Setting up an account with verification of professional credentials using a customID;
Creating user profiles and customizing report settings;
Browsing through their clients' galleries;
Viewing client data (dashboard, photos and treatment regimen);
Viewing, downloading and printing client progress photos and videos;
Accessing client accounts to directly store photos taken during an appointment;
Viewing a client's profile and hair growth regimen, including supplements, medications and topical applications;
Keeping private records for each patient (not shared with client);
Viewing and printing reports on client visits;
Deleting client accounts;
Accessing patient demographic reports;
Creating professional marketing modules to promote their own products and services;
Using marketing e-mail functions to mass e-mail clients or post on their individual account pages;
Advertising their own practice;
Creating team accounts allowing management layers to view their teams' success, progress, and the details of their clients; and
Ability to modify store and logos on their own branded store.

The mobile app 106 is implemented for both iPhone and Android operating systems with Customer Relationship Management (CRM) integration (account creation, helmet registration, chat function, store and customer login). Mobile app 106 can be restricted to only those clients who have purchased a particular brand of laser helmet. Such allows registered users with mobile devices to:
Communicate directly with their hair-loss experts;
Have access to extended SCALPCOACH community 200 functionality;
Have private device storage as well as access to SCALPCOACH community 200 for cloud storage;
Access hair-loss treatment communities to share, ask questions, and join groups to obtain or provide information;
Diagnose their hair-loss level;
Track their treatment progress through photos;
Rate their progress;
Schedule Theradome laser helmet treatments and generate a daily and weekly schedule;
Share updates with hair-loss experts, family and friends through SCALPCOACH web portal 242, email, social media and SCALPCOACH community 200 accounts;
Obtain feedback and guidance from hair-loss professionals;
Compile reports and records from hair-loss professionals;
Obtain notifications and reminders for treatment sessions;
Obtain updates on badges, rewards, blog postings, new products and other Theradome news;
Connect their Theradome Laser Helmet to their mobile device through Bluetooth;
Earn badges and gamification rewards;
Enter monthly contests to win prizes;
Access web portal services through tight integration;
Organize photo galleries and time-lapse videos actual store location should be User Web Portal 260;
Utilize advanced photography tools with templates, ghosting and layering functions; and
Ability to implement other competitive laser products in the future.

A method embodiment of the present invention that improves the results realized by users of hair-loss restoration therapeutic devices begins by delivering therapeutic doses of 678-nm monochromatic laser light to the scalp of a single user from an array of laser diodes inside a helmet. The delivery of therapeutic doses is scheduled according to a treatment regimen with a microcontroller disposed inside the helmet and that has wireless communication with a user's mobile device and a mobile app. Any actual deliveries of the therapeutic doses by the microcontroller are logged and reported through the wireless communication to the user's mobile device and the mobile app to a server. Photographs of the scalp of the single user are periodically taken with the user's mobile device according to a template displayed by the user's mobile device. These are reported as a series of personal progress photos to the server through the user's mobile device and the mobile app. The personal progress photos of the scalp of the single user taken at different times are compared to discern and detect the long-term effectiveness of the delivery of therapeutic doses in the restoration of hair to the scalp of the single user.

A series of the personal progress photos of the scalp of the single user taken at different times is arranged into a time-lapse movie with the server. The single user is reassured as to the effectiveness of a treatment regimen including the delivering of therapeutic doses by displaying the time-lapse movie to the single user though the mobile app to the user's mobile device or through a web portal delivered by the server over the Internet. Reassured users will stay with their treatment regimens and encourage others to buy such devices and subscribe to such treatment regimens.

The actual progress of the single user the effectiveness of a treatment regimen is documented to a professional by displaying the time-lapse movie to the hair-loss professional though a mobile device or through a web portal delivered by the server over the Internet. So, hair-loss professionals armed with such photographic evidence of hair-loss restoration progress or lack of progress are thus better informed in coaching and managing the single users.

Statistics, analyses, and insights are calculated from the logs and personal progress photos reported by many of the single users to the server through their respective mobile devices and mobile apps and making such information selectively available on the Internet with the server.

An important aspect of the Method is connecting together the single user with any of several professionals in a real live voice or texting chat mode. The single user may be rewarded for their commercial loyalty based on usage of the hair-loss restoration therapeutic devices.

Although particular embodiments of the present invention have been described and illustrated, such is not intended to limit the invention. Modifications and changes will no doubt become apparent to those skilled in the art, and it is intended that the invention only be limited by the scope of the appended claims.

1. A method of improving the results realized by users of hair-loss restoration therapeutic devices, comprising:
delivering therapeutic doses of 678-nm monochromatic laser light to the scalp of a single user from an array of laser diodes inside a helmet;
scheduling the delivering of therapeutic doses according to a treatment regimen with a microcontroller disposed inside the helmet and that has wireless communication with a user's mobile device and a mobile app;
logging any actual deliveries of said therapeutic doses by the microcontroller and reporting a log of such through said wireless communication to said user's mobile device and said mobile app to a server;
periodically photographing the scalp of said single user with said user's mobile device according to a template displayed by said user's mobile device, and reporting a series of personal progress photos to said server through said user's mobile device and said mobile app; and
comparing said personal progress photos of the scalp of said single user taken at different times to discern and detect the long-term effectiveness of the delivering therapeutic doses in the restoration of hair to the scalp of the single user.

2. The method of claim 1, further comprising:
arranging a series of said personal progress photos of the scalp of said single user taken at different times into a time-lapse movie with the server.

3. The method of claim 2, further comprising:
reassuring said single user as to the effectiveness of a treatment regimen including the delivering of therapeutic doses by displaying the time-lapse movie to said single user though the mobile app to the user's mobile device or through a web portal delivered by the server over the Internet;
wherein, reassured users stay with their treatment regimens and encourage others to buy such devices and subscribe to such treatment regimens.

4. The method of claim 2, further comprising:
documenting to a hair-loss professional the actual progress of said single user the effectiveness of a treatment regimen including the delivering of therapeutic doses by displaying the time-lapse movie to said hair-loss professional though a mobile device or through a web portal delivered by the server over the Internet;
wherein, hair-loss professionals armed with photographic evidence of hair-loss restoration progress or lack of progress are better informed in coaching and managing said single users.

5. The method of claim 2, further comprising:
building statistics, analyses, and insights from the logs and personal progress photos reported by many of said single users to the server through their respective mobile devices and mobile apps and making such information selectively available on the Internet with the server.

6. The method of claim 1, further comprising:
connecting together said single user with any of several professionals in a real live voice or texting chat mode.

7. The method of claim 1, further comprising:
rewarding said single user for their commercial loyalty based on usage of the hair-loss restoration therapeutic devices.

8. A hair-loss restoration kit of electronics-based therapeutic devices, comprising:
a helmet for delivering therapeutic doses of monochromatic laser light to the scalp of a single user from an array of laser diodes, and that includes a microcontroller and a Bluetooth-type wireless data transceiver;
a mobile device for communication with the Internet and including a Bluetooth-type wireless data transceiver; and
a mobile app installed on the mobile device that controls the Bluetooth-type wireless data transceivers, and that establishes a wireless data communication link between the helmet and the mobile device such that operational data related to the working of the array of laser diodes is accessible to a network server for analysis, reporting, and planning.
